(19) United States
(12) Patent Application Publication
(10) Pub. No.: US 2026/0263113 A1
(43) Pub. Date: Sep. 10, 2026

(54) DILATOR SHAFT AND RELATED METHODS AND KITS

(71) Applicant: Merit Medical Systems, Inc., South Jordan, UT (US)

(72) Inventors: Aaron Hopkinson, Herriman, UT (US); Brendan Knight, Millcreek, UT (US)

(21) Appl. No.: 19/558,149

(22) Filed: Mar. 5, 2026

Related U.S. Application Data
(60) Provisional application No. 63/944,760, filed on Dec. 19, 2025.
(60) Provisional application No. 63/769,509, filed on Mar. 10, 2025.

(51) Int. Cl. A61B 17/34; A61B 17/00; A61M 25/06; A61M 25/09

(52) U.S. Cl. CPC A61B 17/3462; A61B 17/3415; A61B 2017/00526; A61M 25/0662; A61M 25/09041

(57) ABSTRACT

The present disclosure describes a dilator including a body and a shaft secured to the body. The body is configured to detachably secure to one or more of a guide sheath, a microcatheter, and/or an introducer sheath. The shaft is secured to the body and includes a tapered distal end region having a length of about 0.2” to about 0.5”. The shaft includes polyether-ether-ketone material.

### RELATED CASES

This application claims priority to United States Provisional Application No. 63/944,760, filed on December 19, 2025, and titled “DILATOR SHAFT AND RELATED METHODS,” and United States Provisional Application No. 63/769,509, filed on March 10, 2025, and titled “DILATOR SHAFT AND RELATED METHODS,” which are hereby incorporated by reference in their entirety.

### TECHNICAL FIELD

The present disclosure relates generally to medical dilators for insertion of guidewires into vessels.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1A is a perspective view of a dilator secured to an introducer, according to an embodiment.

FIG. 1B is a side view of the dilator of FIG. 1A without the introducer.

FIG. 2A is a side view a tapered distal end region of a shaft of the dilator of FIG. 1B.

FIG. 2B is a partial cross-sectional view of a portion of the dilator, taken through line B-B of FIG. 1B.

FIG. 3 is a flow diagram of a method of manufacturing a shaft for a dilator, according to an embodiment.

### DETAILED DESCRIPTION

This disclosure describes dilators configured to balance stiffness and flexibility to provide an operator access to through dense tissue for successful tracking of a guidewire. In many embodiments, the dilator may include a shaft comprising or consisting essentially of a polyether-ether-ketone (PEEK) material. The PEEK material of the shaft of the dilator allows for a more effective taper of the shaft on a distal end region of the shaft, while avoiding formation of a tip at the distal end that easily snags or is too thin for efficient use. The PEEK material of the shaft of the dilator also may bend more effectively into a vessel for insertion of a guidewire into the vessel. In contrast, conventional shafts of dilators are often too soft, tending to snag, kink, or collapse when attempting to bend the shaft into the vessel or too stiff, leading to unnecessary trauma to the blood vessel (including inadvertent punctures of the blood vessel).

The dilators disclosed herein may be used in combination with guide sheaths, guide wires, microcatheters, and/or introducer sheaths. For example, a physician may insert a dilator of this disclosure into an introducer bore to assist with placing an introducer sheath. The dilator may enter the introducer bore through a seal or a valve that maintains hemostasis when the introducer sheath in in communication with the vasculature. A dilator of this disclosure also may be inserted through a hub and into a sheath shaft of an introducer sheath during use.

The phrase “coupled to” is broad enough to refer to any suitable coupling or other form of interaction between two or more entities, including mechanical, fluidic and thermal interaction. Thus, two components may be coupled to each other even though they are not in direct contact with each other. The phrase “fluid communication” is used in its ordinary sense, and is broad enough to refer to arrangements in which a fluid (e.g., a gas or a liquid) can flow from one element to another element when the elements are in fluid communication with each other.

The terms “proximal” and “distal” are opposite directional terms. As used herein, the distal end of a device or component is the end of the component that is furthest from the physician during ordinary use. The proximal end refers to the opposite end, or the end nearest the physician during ordinary use. For example, the proximal end of an introducer sheath used in minimally invasive vascular treatment is the end accessible to a practitioner during use, while the distal end is disposed within a patient’s vascular system when the sheath is placed into such a patient.

An assembler may be any person, system, or machine used in the manufacture of the dilators.

Embodiments may be understood by reference to the drawings, wherein like parts are designated by like numerals throughout. The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

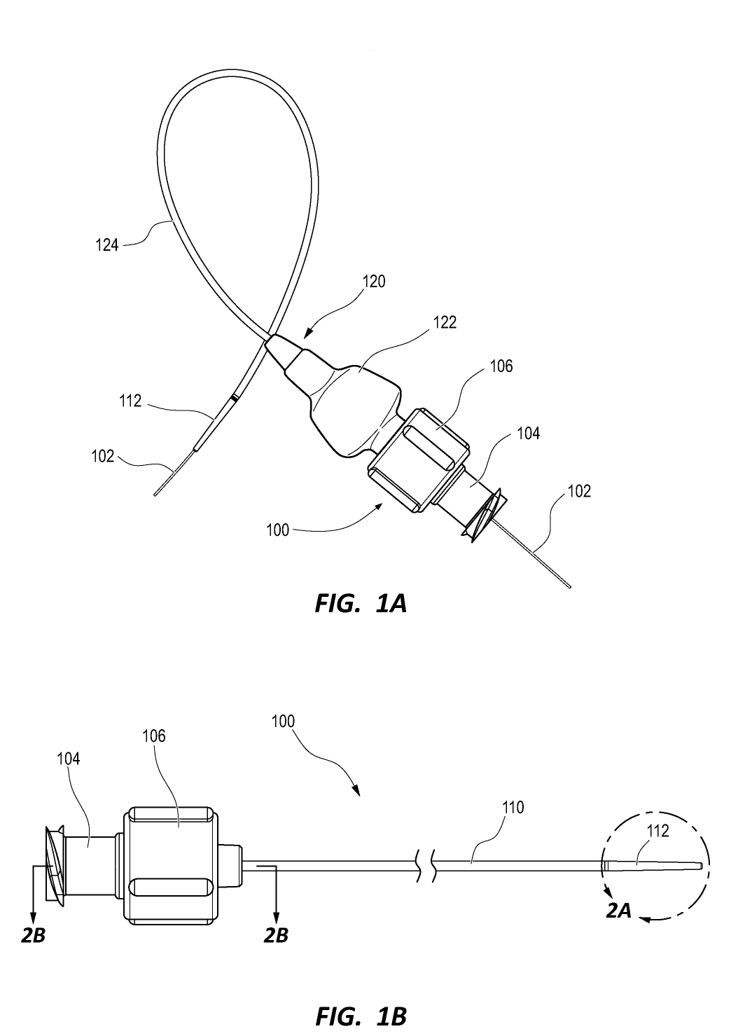

FIG. 1A is a perspective view of an introducer 120 (e.g. microcatheter) secured to a dilator 100, and FIG. 1B is a side view of the dilator 100 without the introducer 120, according to one embodiment. The introducer 120 includes a hub 122 and an introducer sheath 124.

The dilator 100 comprises a shaft 110 having a distal end region 112 that is tapered. In FIG. 1A, generally only the tapered distal end region 112 of the shaft 110 is visible outside the introducer sheath 124 of the introducer 120. The remainder of the shaft 110 is disposed within a lumen of the introducer sheath 124. The tapered distal end region 112 provides a transition between the introducer sheath 124 and the guidewire 102 extending through the tapered distal end region 112 of the dilator 100. This tapered distal end region 112 also may flex and track along the guidewire to position the introducer sheath 124 in a selected and effective position.

The dilator 100 also may include a hub or body secured to the proximal end region of the shaft 110. The hub or body may include one or more (e.g., all) of a luer connector 104 and a fastener 106 secured to the luer connector 104. The dilator 100 may define one or more lumens extending therethrough and sized for insertion and passage of a guidewire 102 through the one or more lumens. The hub or body of the dilator 100 may selectively and detachably secure to the hub 122 of the introducer 120 during insertion into the subject. Once inserted into the subject, the dilator 100 may be uncoupled from the introducer 120, leaving the introducer 120 in place and remove the dilator 100.

The shaft 110 and the distal end region 112 that is tapered may comprise or consist essentially of PEEK material. In some embodiments, the shaft 110 and the distal end region 112 may include PEEK material and one or more of radiopaque fillers, glass-filler material, and/or fiber reinforcement material. In some embodiments, the PEEK material of the shaft 110 and the distal end region 112 may include fluorinated PEEK material. In some embodiments, the shaft 110 and the distal end region 112 comprise polyamide imide, polyimide, acrylic, and/or styrene. With the PEEK material, the shaft 110 and the distal end region 112 may include a substantially golden color (e.g., the PEEK material may result in a hue of gold on the shaft 110 and the distal end region 112). In many embodiments, the shaft 110 and the tapered distal end region 112 include a single, integrally formed member. The shaft 110 and the tapered distal end region 112, for example, may be formed through extrusion of any of the materials (e.g., PEEK material) disclosed herein.

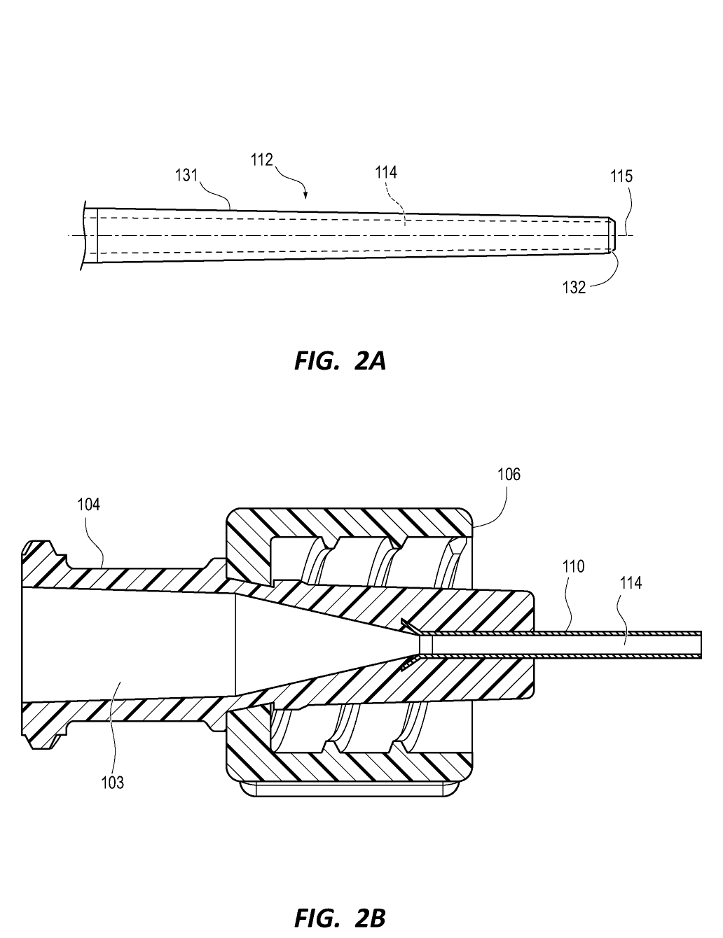

The use of PEEK material in the shaft 110 and the distal end region 112 allows for a much longer tapered region or zone at the distal end region 112. Turning ahead in the drawings, FIG. 2A is a side view the tapered distal end region 112 of the shaft 110 of the dilator 100. The distal end region 112 includes a length extending from the distal end of the shaft 110 to a section of the shaft 110 at which the tapering ends and the outer diameter of the shaft 110 becomes uniform. In the embodiment illustrated in FIG. 2A, the length of the tapered distal end region 112 is approximately 0.363 inches. In other embodiments, the length of the tapered distal end region 112 is at least about 0.2 inch, at least about 0.3 inch, at least about 0.4 inch, about 0.2 inch to about 0.5 inch, such as about 0.2 inch to about 0.3 inch, about 0.3 inch to about 0.4, or about 0.4 inch to about 0.5 inch. The combination of the length, the outer diameter, wall thickness, and the material characteristics of the PEEK material of the distal end region 112 allows the distal end region to bend into a vessel. In many embodiments, when the dilator 100 is secured to a microcatheter, the entire length of the tapered distal end region 112 is positioned outside the microcatheter.

Dilators within the scope of this disclosure may thus be configured to be sufficiently stiff to facilitate introduction into the vessel (e.g., may have sufficient stiffness and pushability to be advanced into the vessel without collapsing, kinking, accordioning, or snagging). Additionally, the dilators may be sufficiently pliable to minimize trauma to the vessel, such as puncturing of the vessel from the inside out by the dilator and/or harming the vessel skiving, scratching, or overly deforming the vessel. Accordingly, the dilators may have both the stiffness necessary to be introduce into the vessel also the pliability necessary to minimize trauma to the vessel. The stiffness characteristics of dilators within the scope of this disclosure may be the “Goldilocks” stiffness, meeting needs for stiffness and flexibility.

In some embodiments, the tapered distal end regions 112 may include one or more tapering portions. For example, the tapered distal end region 112 may include a first or proximal taper portion 131 that is angled less than about 5° from an axis 115 of the distal end region 112. In the embodiment illustrated in FIG. 2A, the first taper portion 131 is angled about 1° from the axis 115. In some embodiments, the first taper portion 131 is angled less than about 4°, less than about 3°, less than about 2°, less than about 1°, about 0.5° to about 5°, about 0.5° to about 1.5°, about 0.75° to about 1.5°, or about 0.75° to about 1.25° from the axis 115 of the distal end region 112.

The tapered distal end region 112 also may include a second or distal taper portion 132 that is angled greater than the first taper portion 131. In the non-limiting embodiment illustrated in FIG. 2A, the second taper portion is angled about 34° from the axis 115 of the distal end region 112. In some embodiments, the second taper portion 132 is angled about 25° to about 45° or about 30° to about 40° from the axis 115 of the distal end region 112.

FIG. 2B is a partial cross-sectional view of a portion of the dilator 100, taken through line B-B of FIG. 1B. The shaft 110 of the dilator 100 may include a substantially uniform outer diameter. In many embodiments, the outer diameter of the shaft 110 may be about 0.05” or less, about 0.04” or less, about 0.03” less, about 0.02” less, such as about 0.012” to about 0.05”, about 0.02 to about 0.042”, about 0.02” to about 0.03”, about 0.03” to about 0.04”, about 0.04” to about 0.05”. In the non-limiting embodiment illustrated in FIG. 2B, the outer diameter of the shaft 110 is about 0.0375”.

As provided above, the shaft 110, including the distal end region 112, defines a lumen 114 extending therethrough. Accordingly, the shaft 110 and the distal end region 112 include an inner diameter defining the lumen 114. In many embodiments, the inner diameter is substantially uniform in both the uniform shaft 110 and the tapered distal end region 112. In the non-limiting embodiment illustrated in FIGS. 2A and 2B, the inner diameter of the shaft 110 and the distal end region 112 is about 0.022”. In some embodiments, the inner diameter of the shaft 110 is about 0.010” to about 0.035”, about 0.012” to about 0.035”, about 0.010” to about 0.020”, about 0.015” to about 0.025”, or about 0.020” to about 0.030”.

In the embodiment of the shaft 110 illustrated in FIG. 2B, the thickness of the wall of the shaft 110 is about 0.0155”. In other embodiments, the thickness of the wall of the shaft 110 may be about 0.005” to about 0.03”, about 0.01” to about 0.025”, or about 0.01” to about 0.02”.

In many embodiments, the shaft 110 and the tapered distal end region 112 of the dilator 100 require significantly less insertion force than conventional dilators. For example, the dilator insertion force of the dilator 100 may be less than about 90 gf, such as about 50 gf to about 80 gf, about 50 gf to about 70 gf, or about 55 gf to about 65 gf.

In many embodiments, the shaft 110 and the tapered distal end region 112 of the dilator 100 have more pushability (e.g., more stiff) than conventional dilators while also retaining the requisite flexibility. For example, the pushability force (e.g., stiffness) of the shaft 110 and tapered distal end region 112 may be about 2.5 N to about 7.5 N, about 4 N to about 6 N, or about 4.5 N to about 5.5 N. In many embodiments, the shaft 110 and the tapered distal end region 112 may have a flexural modulus of about 4 GPa (e.g., about 4.1 GPa).

In many embodiments, the shaft 110 and the tapered distal end region 112 of the dilator 100 are more trackable than conventional dilators. Effective trackability and flexibility of the shaft 110 and the tapered distal end region 112 allow the dilator 100 to flex and track on the guidewire 102. A three-point bend test may be used to infer the flexibility/trackability of the dilator. According to some aspects, a three-point bend of the shaft 110 and the tapered distal end region 112 of the dilator 100 indicate a trackability of about 0.040 N to about 0.050 N or about 0.042 N to about 0.046 N.

As further shown in FIG. 2B, the shaft 110 may be secured to the connector 104 with the lumen 114 of the shaft 110 in fluid communication with a conduit 103 of the connector 104. For example, the shaft 110 may be secured to a head of the connector 104. The fastener 106 may be secured to the connector 104 between the distal head and proximal threading of the fastener 106. The fastener 106 may include internal threading configured to threadedly engage with male threading. The fastener 106 may be configured to detachably and selectively secure to the hub 122 of the introducer sheath 120.

Also disclosed herein is an introducer kit comprising an introducer 120 and the dilator 100. The introducer 120 in the introducer kit may comprise the hub 122 and the introducer sheath 124 secured to the hub 120. The dilator 100 in the introducer kit may comprise any dilator disclosed herein. For example, the dilator 100 in the introducer kit may comprise a body configured to detachably secure to the introducer sheath 124 and a shaft 100 secured to the body. The shaft 100 may comprise a distal end region 112 with a tapered portion having a length of about 0.2” to about 0.5”. The shaft 100 of the dilator in the introducer kit may be sized and dimensioned such that the tapered portion of the distal end region 112 extends from the introducer sheath 124 of the introducer 120 when the dilator 100 is secured to the introducer 120.

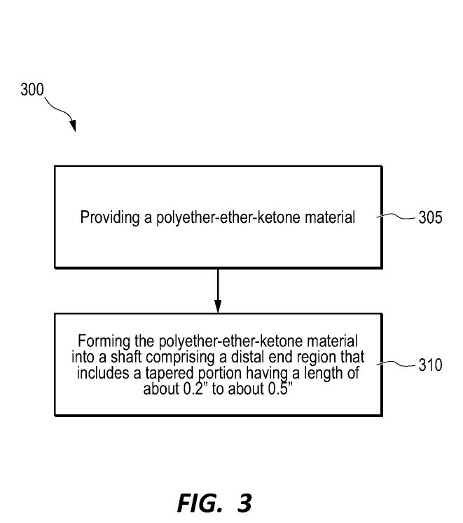

Also disclosed herein are methods of manufacturing a shaft for a dilator and methods of assembling a dilator. FIG. 3, for example, is a flow diagram of a method 300 of manufacturing a shaft for a dilator, according to an embodiment. Embodiments of the method 300 may form any of the shafts or dilators disclosed herein. The method 300 may include providing 305 a polyether-ether-ketone material and forming 310 the polyether-ether-ketone material into a shaft comprising a distal end region that is tapered for a length of about 0.2” to about 0.5”.

In many embodiments, forming 310 the polyether-ether-ketone material into the shaft may comprise extruding the polyether-ether-ketone material into an elongated tube having a substantially uniform wall thickness, cutting the elongated tube at one or more locations to form the shaft having a proximal end region and the distal end region, and tapering the distal end region of the shaft. In some embodiments of the method 300, the shaft having the proximal end region and the distal end region may be formed without cutting the elongated tube (e.g., the elongated tube may be extruded at a predetermined dimension of the shaft). In some embodiments of the method 300, the elongated tube and/or shaft may be formed in any suitable method that does not require extruding.

In many embodiments, tapering the distal end region comprises disposing the distal end region of the shaft within a tapered region of a die and heating the distal end region of the shaft while the distal end region is disposed within the tapered region of the die. The tapered region of the die may include a chamber or bore sized complementary to the predetermined shape and/or length of the tapering of the distal end region of the shaft. In many embodiments, heating the distal end region may comprise applying a radio frequency energy to the distal end region of the shaft while the distal end region is disposed within the tapered region of the die. Other embodiments may include any suitable application of heat to the distal end region of the shaft.

In some embodiments, the material extruded to form the shaft consists essentially of the polyether-ether-ketone material. In many embodiments, the method may comprise mixing the polyether-ether-ketone material with one or more of glass-filler material, fiber-reinforcement material, or fluorinated polyether-ether-ketone material. The method also may include mixing the polyether-ether-ketone material with at least one of polyamide imide material or polyimide material.

The shaft formed according to embodiments of the method 300 disclosed herein may include any of the dimensions provided above. For example, the length of the taper at the distal end region may be about 0.035” to about 0.04”, the shaft may comprise an outer diameter of about 0.0180” to about 0.050”, the shaft may define a lumen having an inner diameter of about 0.012” to about 0.035”, and the shaft may comprise a wall defining the lumen and having a thickness of about 0.01” to about 0.02”.

The method 300 of manufacturing a dilator also may include securing the shaft formed according to the method provided above to a body. For example, the method may include securing the shaft to the fastener.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “near.” For each such reference, it is to be understood that, in some embodiments, the value, feature, or characteristic may be specified without approximation. For example, where qualifiers such as “near” and “approximately” are used, these terms include within their scope the qualified words in the absence of their qualifiers. For example, where the term “approximately aligned” is recited with respect to a feature, it is understood that in further embodiments, the feature can have a precisely aligned configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A dilator, comprising:
a body configured to detachably secure to an introducer sheath; and
a shaft secured to the body and comprising a distal end region with a tapered portion having a length of about 0.2” to about 0.5”, wherein the shaft is sized and dimensioned such that the tapered portion of the distal end region extends from an introducer sheath of an introducer when the dilator is secured to the introducer.

2. The dilator of claim 1, wherein the shaft comprises polyether-ether-ketone material.

3. The dilator of claim 2, wherein the polyether-ether-ketone material further comprises one or more of glass-filler material, fiber-reinforcement material, or fluorinated polyether-ether-ketone material.

4. The dilator of claim 2, wherein the shaft consists essentially of polyether-ether-ketone material.

5. The dilator of claim 1, wherein the shaft comprises at least one of polyamide imide material or polyimide material.

6. The dilator of claim 1, wherein the length of the tapered portion is about 0.035” to about 0.04”.

7. The dilator of claim 1, wherein the shaft comprises an outer diameter of about 0.0180” to about 0.050”.

8. The dilator of claim 1, wherein the shaft defines a lumen having an inner diameter of about 0.012” to about 0.035”.

9. The dilator of claim 8, wherein the shaft comprises a wall defining the lumen and having a thickness of about 0.01” to about 0.02”.

10. A method of manufacturing a shaft for a dilator, the method comprising:
providing a polyether-ether-ketone material; and
forming the polyether-ether-ketone material into a shaft comprising a distal end region that includes a tapered portion having a length of about 0.2” to about 0.5”.

11. The method of claim 10, wherein forming the polyether-ether-ketone material into the shaft comprises:
extruding the polyether-ether-ketone material into an elongated tube having a substantially uniform wall thickness;
cutting the elongated tube at one or more locations to form the shaft having a proximal end region and the distal end region; and
tapering the distal end region of the shaft to form the tapered portion.

12. The method of claim 11, wherein tapering the distal end region comprises:
disposing the distal end region of the shaft within a tapered region of a die; and
heating the distal end region of the shaft while the distal end region is disposed within the tapered region of the die to form the tapered portion.

13. The method of claim 12, wherein heating the distal end region comprises applying a radio frequency energy to the distal end region of the shaft while the distal end region is disposed within the tapered region of the die to form the tapered portion.

14. An introducer kit, comprising:
an introducer comprising a hub and an introducer sheath secured to the hub; and
a dilator comprising a body configured to detachably secure to the introducer sheath and a shaft secured to the body, the shaft comprising a distal end region with a tapered portion having a length of about 0.2” to about 0.5”, wherein the shaft is sized and dimensioned such that the tapered portion of the distal end region extends from the introducer sheath of the introducer when the dilator is secured to the introducer.

15. The introducer kit of claim 14, wherein the shaft comprises polyether-ether-ketone material.

16. The introducer kit of claim 15, wherein the polyether-ether-ketone material further comprises one or more of glass-filler material, fiber-reinforcement material, or fluorinated polyether-ether-ketone material.

17. The introducer kit of claim 15, wherein the shaft consists essentially of polyether-ether-ketone material.

18. The introducer kit of claim 14, wherein the shaft comprises at least one of polyamide imide material or polyimide material.

19. The introducer kit of claim 14, wherein the length of the tapered portion is about 0.035” to about 0.04”.

20. The introducer kit of claim 14, wherein the shaft comprises an outer diameter of about 0.0180” to about 0.050”.
